(19) United States
(12) Patent Application Publication
(10) Pub. No.: US 2009/0048214 A1
(43) Pub. Date: Feb. 19, 2009

(54) Methods for Treating Renal Tumors Using 2, 4-Pyrimidinediamine Drug and Prodrug Compounds

(75) Inventors: Yasumichi Hitoshi, Brisbane, CA (US); Elliott Grossbard, San Francisco, CA (US); Ankush Argade, Foster City, CA (US); Rajinder Singh, Belmont, CA (US); Hui Li, Santa Clara, CA (US); David Carroll, San Francisco, CA (US); Susan Catalano, Hayward, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: RIGEL PHARMACEUTICALS, INC, South San Francisco, CA (US)

(21) Appl. No.: 11/940,930

(22) Filed: Nov. 15, 2007

Related U.S. Application Data
(60) Provisional application No. 60/866,061, filed on Nov. 15, 2006.

(51) Int. Cl. A61K 31/5383; A61P 35/00; A61K 31/675

(52) U.S. Cl. 514/81; 514/230.5

(57) ABSTRACT

The present disclosure provides methods for the inhibiting proliferation of tumor cells, and methods for treating solid tumor cancers in a subject by administration of 2,4-pyrimidinediamine compounds.

### 1. CROSS REFERENCE TO RELATED APPLICATIONS

This application claims the benefit of U.S. provisional application No. 60/866,061, filed Nov. 15, 2006, hereby incorporated by reference in its entirety.

### 2. BACKGROUND OF THE INVENTION

2.1 Field of the Invention

The present disclosure concerns methods of inhibiting proliferation of tumor cells and treating solid tumor cancers using certain 2,4 pyrimidinediamine compounds or prodrugs thereof.

2.2 Description of Related Art

Cancer is a group of varied diseases characterized by uncontrolled, abnormal growth and division of cells. Cancer cells typically bear one or more abnormalities in the molecular mechanisms that control of cell growth and division, such as cell cycle checkpoint controls or signaling pathways involved in cellular communication. Through successive rounds of mutation and natural selection, a group of abnormal cells, generally originating from a single mutant cell, accumulates additional mutations that provide selective growth advantage over other cells, and thus evolves into a cell type that predominates in the cell mass and continues to divide unchecked. The process of mutation and natural selection is enhanced by genetic instability displayed by many types of cancer cells, an instability which is gained either from somatic mutations or by inheritance from the germ line. The enhanced mutability of cancerous cells increases the probability of their progression towards formation of malignant cells. As the cancer cells further evolve, cells from the resulting cell mass, or tumor, may become locally invasive and may spread through the blood or lymph to start new cancers in tissues other than the cancer cell's tissue of origin (metastases), colonizing and destroying surrounding normal tissues. This property along with the heterogeneity of the tumor cell population makes cancer a particularly difficult disease to treat and eradicate.

Traditional cancer treatments take advantage of the higher proliferative capacity of cancer cells and their increased sensitivity to DNA damage. Ionizing radiation, including γ-rays and x-rays, and cytotoxic agents, such as bleomycin, cisplatin, vinblastine, cyclophosphamide, 5′-fluorouracil, and methotrexate rely upon a generalized damage to DNA or block DNA synthesis mechanisms, destabilizing chromosomal structure and eventually leading to destruction of cancer cells. These treatments are particularly effective for those types of cancers that have defects in the cell cycle checkpoint, because such defects limit the ability of these cells to repair damaged DNA, or to properly replicate DNA before undergoing cell division. The non-selective nature of these treatments, however, often results in severe and debilitating side effects. The systemic use of these drugs may result in damage to normally healthy organs and tissues, and compromise the long term health of the patient.

Although more selective chemotherapeutic treatments have been developed based on knowledge of how cancer cells develop, for example, the anti-estrogen compound tamoxifen, the effectiveness of all chemotherapeutic treatments is subject to development of resistance to the drugs. In particular, the increased expression of cell membrane bound transporters, such as MdrI, produces a multidrug resistance phenotype characterized by increased efflux of drugs from the cell. These types of adaptation by cancer cells severely limit the effectiveness of certain classes of chemotherapeutic agents.

Renal cell carcinoma is the sixth leading cause of cancer death, and is characterized by a lack of early warning signs, diverse clinical manifestations, resistance to radiation and chemotherapy, and infrequent but reproducible responses to immunotherapy agents such as interferon alpha and interleukin (IL)-2. Consequently, identification of other chemotherapeutic agents is critical for establishing therapies effective for attacking the heterogeneous nature of proliferative diseases such as cancer and for overcoming any resistance that may develop over the course of therapy with other compounds. Moreover, use of combinations of chemotherapeutic agents with differing properties and cellular targets increases the effectiveness of chemotherapy and limits the generation of drug resistance.

### 3. SUMMARY OF THE INVENTION

It has been discovered that certain 2,4-pyrimidinediamine compounds are potent inhibitors of proliferation of abnormal cells, such as tumor cells, in in vitro assays. In particular, these compounds have demonstrated potent inhibition against renal tumor cell lines and others. The compounds can therefore be used to inhibit proliferation of tumor cells in vitro and in vivo in a variety of contexts. Prodrugs of the compounds that yield the active drug compound under the conditions of use can also be used to inhibit tumor cell proliferation in a variety of in vitro and in vivo contexts.

Accordingly, in one aspect, the present disclosure provides methods of inhibiting proliferation of tumor cells. The method generally involves administering to a tumor cell an amount of a 2,4-pyrimidinediamine drug compound, or an acceptable salt, hydrate, solvate and/or N-oxide thereof, effective to inhibit proliferation of the tumor cell. The method may be practiced in in vitro contexts or in in vivo contexts as a therapeutic approach towards the treatment or prevention of proliferative disorders, such as tumorigenic cancers.

The drug compound can be administered to the cells, or alternatively, the drug compound can be provided by administration of a prodrug form of the drug compound. When administered as a prodrug, the methods may be carried out under conditions in which the prodrug compound converts into the active drug compound.

The mode of administration will depend upon the context of the method. For methods carried out in vitro, administration can be effected by contacting the cells with the drug or prodrug. For methods carried out in vivo, administration can be effected by administration of the prodrug, and physiological conditions can convert the prodrug to the drug form.

Drug compounds useful in the methods are generally 2,4-pyrimidinediamine compounds, exemplified by Compound 1:

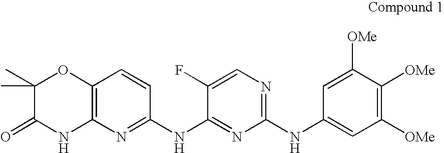

including salts, hydrates, and N-oxides thereof.

In some embodiments, the tumor cell is a renal tumor cell.

In some embodiments, the method is carried out in vitro. In other embodiments, the method is carried out in vivo in a subject. In some embodiments, the drug compound is supplied in the form of a prodrug compound, and the method is carried out under conditions in which the prodrug compound yields the drug compound.

Also useful are prodrugs of the 2,4-pyrimidinediamine drug compounds. Such prodrugs may be active in their prodrug form, or may be inactive until converted under physiological or other conditions of use to an active drug form. In the prodrugs, one or more functional groups of the 2,4-pyrimidinediamine compounds are included in promoieties that cleave from the molecule under the conditions of use, typically by way of hydrolysis, enzymatic cleavage or some other cleavage mechanism, to yield the functional groups.

The prodrugs useful in the methods described herein are generally substituted at a nitrogen atom of one or more primary or secondary amine groups with a progroup R that metabolizes or otherwise transforms under conditions of use to yield the active 2,4-pyrimidinediamine drug compound. In some embodiments, the progroup R is a phosphorous-containing progroup that includes a phosphate moiety that can be cleaved in vitro or in vivo spontaneously, such as by way of a hydrolysis reaction, or cleavage may be catalyzed or induced by another agent, such as by endogenous or exogenous enzymes (for example, esterases, lipases and/or phosphatases), by acidic or basic conditions, by light, or by a change of exposure to a physical or environmental parameter, such as a change of temperature. The agent may be endogenous to the conditions of use, such as an enzyme present in the cells to which the prodrug is administered or the acidic conditions of the stomach, or it may be supplied exogenously. Endogenous enzymes are prevalent throughout the body, residing in, for example, the stomach and digestive tract, blood and/or serum, and in virtually all tissues and organs. Such phosphate-containing progroups R will generally increase the water-solubility of the underlying active 2,4-pyrimidinediamine drug compound, making such phosphate-containing prodrugs ideally suited for modes of administration where water-solubility is desirable, such as, for example, oral, buccal, intravenous, intramuscular and ocular modes of administration to a subject (preferably a human subject). Solubility and bioavailability characteristics of specific 2,4-pyrimidinediamine drugs and prodrugs described herein are detailed in U.S. application Ser. No. 11/337,049 filed Jan. 19, 2006 (US2006/0211657 A1), at paragraphs 26-27 and 112-114 of the printed publication, which paragraphs are hereby incorporated by reference.

Thus, also provided herein is a method of inhibiting the proliferation of a tumor cell comprising administering to a tumor cell a prodrug compound according to structural formula (I), in an amount effective to, and under conditions suitable to, yield an amount of a drug compound effective to inhibit proliferation of the tumor cell:

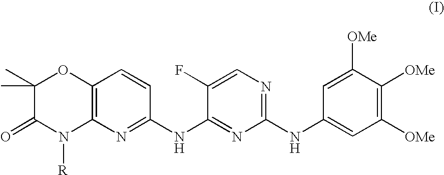

including salts, hydrates, and/or N-oxides thereof, wherein
R represents a progroup.

In some embodiments, the progroup includes a group or moiety that is metabolized under the conditions of use to yield an unstable α-hydroxymethyl, α-aminomethyl or α-thiomethyl intermediate, which is then further metabolized in vivo to yield the active 2,4-pyrimidinediamine drug. In some embodiments, the progroup may be, but is not limited to, an acid labile hydroxyalkyl-containing progroup, an acid labile thio containing progroup, an acid labile amino containing progroup, an acid labile phosphate containing progroup, and salts thereof.

In some embodiments, the progroup is —CH₂—O—P(O)(OH)₂, including ionized forms, e.g., —CH₂—O—P(O)(OH)O⁻ or —CH₂—O—P(O)(O⁻)₂, or salts thereof.

In another aspect, the present disclosure provides methods of treating or preventing cancers in subjects, such as solid tumor cancers. The methods generally comprise administering to the subject an amount of a compound according to structural formula (II) effective to treat or prevent the cancer:

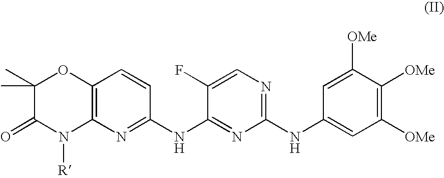

including salts, hydrates, and/or N-oxides thereof,
wherein R′ is selected from hydrogen and a progroup.

Specific examples of progroups R′ include those discussed below.

The drug or prodrug compounds may be administered to the subject as the compounds, per se, or in the form of pharmaceutical compositions. The exact form of pharmaceutical composition may depend, in part, on the mode of administration, which can range from virtually any mode of administration to a subject, including, but not limited to, topical, ocular, oral, buccal, systemic, nasal, injection, transdermal, rectal, vaginal, etc., forms of administration, or by administration by inhalation or insufflation.

The methods may be practiced in animals in a veterinary context, including, but not limited to, bovine, equine, feline, canine or rodent animals, or in humans, and may be practiced alone as monotherapy, or in combination with, or adjunct to, other cancer therapies, such as in combination with other chemo- or radiation-therapies, or adjunct to removal of the tumor by surgery.

The methods are useful for treating and/or preventing a wide range of tumorigenic and other cancers, such as carcinomas, sarcomas, leukemias, and cancers of the nervous system.

In some embodiments, the tumorigenic cancers treated or prevented are renal cancers, including, but not limited to, renal cell carcinoma, clear cell carcinoma of kidney, and renal cell adenocarcinoma. In some embodiments, the solid tumor cancer is renal cell carcinoma and/or renal cell adenocarcinoma. The tissue of origin for renal cell carcinoma is the proximal renal tubular epithelium. Renal cancer occurs in both a sporadic (nonhereditary) and a hereditary form, and both forms are associated with structural alterations of the short arm of chromosome 3 (3p). Genetic studies of the families at high risk for developing renal cancer led to the cloning of genes whose alteration results in tumor formation. These genes are either tumor suppressors (VHL, TSC) or oncogenes (MET). At least 4 hereditary syndromes associated with renal cell carcinoma are recognized: (1) von Hippel-Lindau (VHL) syndrome, (2) hereditary papillary renal carcinoma (HPRC), (3) familial renal oncocytoma (FRO) associated with Birt-Hogg-Dube syndrome (BHDS), and (4) hereditary renal carcinoma (HRC).

### 4. DESCRIPTION OF DRAWINGS

FIG. 1. In Vivo Evaluation of Compound A on A498 Tumor Growth in NCR nu/nu Mice. Test compound was administered ad libitum in the feed as a formulation of 0, 0.5, 2.0, or 3.0 g of Compound A per kg of AIN-76A rodent diet. Mean tumor volume was 82 mm³ on Day 0.

FIG. 2. In Vivo Evaluation of Compound A on the Body Weight of A498 Tumored Animals. Test compound was administered ad libitum in the feed as a formulation of 0, 0.5, 2.0, or 3.0 g of Compound A per kg of AIN-76A rodent diet.

FIG. 3. Tumor Growth Curve Slope Evaluation of A498 Tumors in NCR nu/nu Mice. Test compound was administered ad libitum in the feed as a formulation of 0, 0.5, 2.0, or 3.0 g of Compound A per kg of AIN-76A rodent diet.

FIG. 4. Median Tumor Weight of RXF-393 Renal Carcinomas Implanted Orthotopically in Nude Mice After Treatment with Compound A. Test compound was administered in the feed as a formulation of 0, 75, 300, or 450 mg of Compound A per kg per dose of AIN-76A rodent diet. The doses were calculated to be the total of 0, 0.5, 2.0, or 3.0 g of Compound A per kg.

FIG. 5. In Vivo Evaluation of Compound A on the Body Weight of Animals Implanted with RXF-393 Renal Carcinoma. Test compound was administered ad libitum in the feed as a formulation of 0, 75, 300, or 450 g of Compound A per kg per dose of AIN-76A rodent diet.

### 5. DETAILED DESCRIPTION

5.1 Definitions

As used herein, the following terms are intended to have the following meanings:

“Alkyl” by itself or as part of another substituent refers to a saturated or unsaturated branched, straight-chain or cyclic monovalent hydrocarbon radical having the stated number of carbon atoms (i.e., C₁-C₆ means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. As used herein, “lower alkyl” means (C₁-C₈) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. As used herein, “lower alkanyl” means (C₁-C₈) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. As used herein, “lower alkenyl” means (C₂-C₈) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. As used herein, “lower alkynyl” means (C₂-C₈) alkynyl.

“Heteroalkyl,” “Heteroalkanyl,” “Heteroalkenyl,” “Heteroalkynyl,” “Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C₁-C₈) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Aryl” by itself or as part of another substituent refers to a monovalent aromatic hydrocarbon group having the stated number of carbon atoms (i.e., C₆-C₁₅ means from 6 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C₆-C₁₅) aryl, with (C₆-C₁₀) being more typical. Specific exemplary aryls include phenyl and naphthyl.

“Arylalkyl” by itself or as part of another substituent refers to an acyclic alkyl group in which one of the hydrogen atoms bonded to a carbon atom, typically a terminal or sp.sup.3 carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In some embodiments, the arylalkyl group is (C₇-C₂₁) arylalkyl, e.g., the alkanyl, alkenyl or alkynyl moiety of the arylalkyl group is (C₁-C₆) and the aryl moiety is (C₆-C₁₅). In some specific embodiments the arylalkyl group is (C₇-C₁₃), e.g., the alkanyl, alkenyl or alkynyl moiety of the arylalkyl group is (C₁-C₃) and the aryl moiety is (C₆-C₁₀).

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuiran, benzopyrone, benzothiadiazole, benzothiazole, benzotriazole, benzoxazine, benzoxazole, benzoxazoline, carbazole, .beta.-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroarylalkyl” by itself or as part of another substituent refers to an acyclic alkyl group in which one of the hydrogen atoms bonded to a carbon atom, typically a terminal or sp.sup.3 carbon atom, is replaced with a heteroaryl group. Where specific alkyl moieties are intended, the nomenclature heteroarylalkanyl, heteroarylakenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In some specific exemplary embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR″′, where R″′ is a haloalkyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3.sup.rd Ed., 1999, John Wiley & Sons, NY and Harrison et al., Compendium of Synthetic Organic Methods, Vols. 1-8, 1971-1996,John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl and trityl ethers, as well as alkyl ethers, tetrahydropyranyl ethers, trialkylsilyl ethers (e.g., TMS or TIPPS groups) and allyl ethers.

“Substituted,” when used to modify a specified group or radical, means that one or more hydrogen atoms of the specified group or radical are each, independently of one another, replaced with the same or different substituent(s). Substituent groups useful for substituting for hydrogens on saturated carbon atoms in the specified group or radical include, but are not limited to —R⁶⁰, halo, —O⁻M⁺, ═O, —OR⁷⁰, —SR⁷⁰, —S⁻M⁺, ═S, —NR⁸⁰R⁸⁰, ═NR⁷⁰, ═N—OR⁷⁰, trihalomethyl, —CF₃, —CN, —OCN, —SCN, —NO, —NO₂, ═N₂, —N₃, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, (—C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰C(O)O⁻M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of alkyl, cycloalkyl, heteroalkyl, cycloheteroalkyl, aryl, arylalkyl, heteroaryl and heteroarylalkyl; each R⁷⁰ is independently hydrogen or R⁶⁰; each R⁸⁰ is independently R⁷⁰ or alternatively, the two R⁸⁰'s, taken together with the nitrogen atom to which they are bonded, form a 5-, 6- or 7-membered cycloheteroalkyl which may optionally include from 1 to 4 of the same or different additional heteroatoms selected from the group consisting of O, N and S; and each M⁺ is a counter ion with a positive charge, for example, a positive charge independently selected from K⁺, Na⁺, ⁺N(R⁶⁰)₄, and Li⁺, or two of M⁺, combine to form a divalent counterion, for example a divalent counterion selected from Ca²⁺, Mg²⁺, and Ba²⁺. As specific examples, —NR⁸⁰R⁸⁰ is meant to include —NH₂, —NH-alkyl, N-pyrrolidinyl and N-morpholinyl.

Similarly, substituent groups useful for substituting for hydrogens on unsaturated carbon atoms in the specified group or radical include, but are not limited to, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻—)₂(M⁺)₂, —P(O)(OR⁷⁰)O⁻M⁺, —P(O)(OR⁷⁰)(OR⁷⁰), —C(O)R⁷⁰, —C(S)R⁷⁰, —CNR⁷⁰)R⁷⁰, —C(O)O⁻—M⁺, —C(O)R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰C(O)O⁻M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R³⁰ and M⁺ are as previously defined.

Substituent groups useful for substituting for hydrogens on nitrogen atoms in heteroalkyl and cycloheteroalkyl groups include, but are not limited to, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups from the above lists useful for substituting other groups or atoms specified as “substituted” will be apparent to those of skill in the art.

“Cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder may have the same cell division rates as normal cells but do not respond to signals that limit such growth. Within the ambit of “cell proliferative disorder” is neoplasm or tumor, which is an abnormal growth of tissue. Cancer refers to any of various malignant neoplasms characterized by the proliferation of cells that have the capability to invade surrounding tissue and/or metastasize to new colonization sites.

“Inhibition of proliferation” refers to an arrest of cell division, a reduction in the rate of cell division, proliferation and/or growth, and/or induction of cell death. The drugs or prodrugs disclosed herein have been shown to inhibit the proliferation of treated cells as compared to an untreated control cells of a similar type. As used herein, inhibition of proliferation can be brought about by any mechanism or combination of mechanisms, and may operate to inhibit proliferation cytostatically or cytotoxically.

“GI₅₀” refers to the concentration of compound at which inhibition of growth of 50% of the population of cells being assayed is observed.

“TGI” refers to the concentration of compound at which total inhibition of growth of cells being assayed is observed.

“LC₅₀” refers to the concentration of compound which results in lethality in 50% of the population of cells being assayed.

5.2 The Drug Compounds

Drug compounds useful in the methods are generally 2,4-pyrimidinediamine compounds, as exemplified by Compound 1:

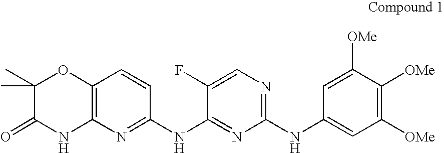

including salts, hydrates, and N-oxides thereof.

5.3 The Prodrug Compounds

Prodrugs are derivatives of drug compounds that require transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

The class of 2,4-pyrimidinediamine drug and prodrug compounds has been previously described in detail in U.S. application Ser. No. 11/337,049 filed Jan. 19, 2006 (US2006/0211657), and U.S. application Ser. No. 10/913,270 filed Aug. 6, 2004 (US2005/0113398), the disclosures of which are incorporated herein by reference in their entirety.

Prodrug compounds useful in the methods described herein are generally 2,4-pyrimidinediamine compounds according to structural formula (I), administered in an amount effective to, and under conditions suitable to, yield an amount of a drug compound effective to inhibit proliferation of tumor cells:

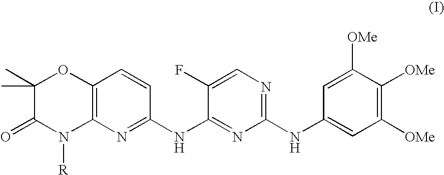

including salts, hydrates, and/or N-oxides thereof, wherein R represents a progroup.

A progroup can include, but is not limited to, a group or moiety that is metabolized under the conditions of use to yield an unstable α-hydroxyalkyl, α-aminoalkyl or α-thioalkyl intermediate (for example, α-hydroxymethyl, α-aminomethyl or α-thiomethyl intermediate), which then further metabolized in vivo to yield the active 2,4-pyrimidinediamine drug. In some embodiments, the progroup may be, but is not limited to, an acid labile hydroxyalkyl-containing progroup, an acid labile thio containing progroup, an acid labile amino containing progroup, an acid labile phosphate containing progroup, and salts thereof. Each of the acid labile thio containing progroup and the acid labile amino containing progroup may be thioalkyl and aminoalkyl groups, respectively. In some embodiments the acid labile hydroxyalkyl-containing progroup, acid labile thio containing progroup, and an acid labile amino containing progroup may be capped as the corresponding phosphate, e.g., —CH₂—O—P(O)(OH)₂, thiophosphate e.g. —CH₂—S—P(O)(OH)₂, and phosphoramidate e.g. —CH₂—NH—P(O)(OH)₂, respectively, to make prodrug groups. These prodrug groups can be free acids as depicted, alkyl esters, or salts, e.g. metal salts, and combinations thereof.

In some embodiments, the progroup R is of the formula —CR¹R¹-A-R³, where each R¹ is independently selected from hydrogen, cyano, —C(O)R², —C(O)OR², C(O)NR²R², —C(OR²)(OR²), optionally substituted (C₁-C₂₀) alkyl, (C₁-C₂₀) haloalkyl, optionally substituted (C₆-C₁₄) aryl, optionally substituted (C₇-C₃₀) arylalkyl, optionally substituted 5-15 membered heteroaryl, and optionally substituted 6-30 membered heteroarylalkyl, where each R² is independently selected from hydrogen, (C₁-C₈) alkyl, aryl (for example phenyl or naphthyl, arylalkyl such is benzyl), heteroaryl, and heteroarylalkyl; A is selected from O, S, and NR⁴, where R⁴ is selected from R¹ and cycloalkyl, or, alternatively, is taken together with R³ such that R⁴ and R³, together with nitrogen atom to which they are attached, form a three- to seven-membered ring; and R³ is a group that, together with A, metabolizes under the conditions of use to yield an intermediate group of the formula —CR¹R¹AH.

In one embodiment, the progroup R is of the formula —CR¹R¹-A-R³, where each R¹ is independently selected from hydrogen, optionally substituted lower alkyl, optionally substituted (C₆-C₁₄) aryl, and optionally substituted (C₇-C₂₀) arylalkyl; where the optional substituents are, independently of one another, selected from hydroxyl, lower alkoxy, (C₆-C₁₄) aryloxy, lower alkoxyalkyl, methoxymethyl, methoxyethyl, ethoxymethyl, ethoxyethyl and halogen, or, alternatively, two R¹ bonded to the same carbon atom are taken together with the carbon atom to which they are bonded to form a cycloalkyl group containing from 3 to 8 carbon atoms; A is selected from O, S and NR⁴, where R⁴ is selected from hydrogen, alkyl, aryl, arylalkyl, heteroaryl, heteroarylalkyl and cycloheteroalkyl, or alternatively is combined with R³, and, together with the nitrogen to which they are attached, form a three to seven membered ring; and R³ represents a group that can be metabolized in vivo to yield a group of the formula —CR¹R¹AH.

The mechanism by which the R group metabolizes to yield intermediate group —CR¹R¹-A-H is not critical, and can be caused by, for example, hydrolysis under the acidic conditions of the stomach, and/or by enzymes present in the digestive tract and/or tissues or organs of the body. Indeed, the R group(s) can be selected to metabolize at a particular site within the body. For example, many esters are cleaved under the acidic conditions found in the stomach. Prodrugs designed to cleave chemically in the stomach to the active 2,4-pyrimidinediamine can employ progroups including such esters. Alternatively, the progroups may be designed to metabolize in the presence of enzymes such as esterases, amidases, lipolases, phosphatases including ATPases and kinase etc., to yield the intermediate group of formula —CR¹R¹-A-H. Progroups including linkages capable of metabolizing in vivo to yield such an intermediate group are well-known, and include, by way of example and not limitation, ethers, thioethers, silylethers, silylthioethers, esters, thioesters, carbonates, thiocarbonates, carbamates, thiocarbamates, ureas, thioureas, carboxamides, etc. In some instances, a “precursor” group that is oxidized by oxidative enzymes such as, for example, cytochrome P450 of the liver, to a metabolizable group, can be selected.

The identity of the R group can also be selected so as to impart the prodrug with desirable characteristics. For example, lipophilic groups can be used to decrease water solubility and hydrophilic groups can be used to increase water solubility. In this way, prodrugs specifically tailored for selected modes of administration can be obtained. The R group can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, targeting-specific transporters, etc. Groups capable of imparting prodrugs with these characteristics are well-known, and are described, for example, in Ettmayer et al., 2004, J. Med. Chem. 47(10:2393-2404), the disclosure of which is incorporated by reference. All of the various groups described in these references can be utilized in the prodrugs described herein.

In some embodiment, R³ includes, together with A, an ether, a thioether, a silyl ether, a silyl thioether, an ester, a thioester, an amide, a carbonate, a thiocarbonate, a carbamate, a thiocarbamate, or a urea linkage, —OCH₂SO₃R, where R is hydrogen, alkyl, aryl, arylalkyl or a metal salt (e.g., sodium, lithium, potassium); -GCH₂⁺N(R⁵¹)₃M⁻, where G is absent, —OPO⁻, OSO₃— or —CO₂—, R₅₁ is hydrogen, alkyl, aryl, arylalkyl, cycloheteroalkyl or cycloheteroalkylalkyl and M- is a counterion, usually a halide ion or the like (acetate, sulfate, phosphate, etc.). In other embodiments, R³ is selected from —R⁵, —C(O)R⁵, —C(O)NR⁵R⁵ and —SiR⁵R⁵R⁵, where the R⁵ groups are selected so as to impart the prodrugs with desired bioavailability, cleavage and/or targeting properties. In a specific embodiment, the R⁵ groups are selected to impart the prodrug with higher water-solubility than the underlying active 2,4-pyrimidinediamine drug. Thus, in some embodiments, the R⁵ groups are selected such that they, taken together with the heteroatom or group to which they are bonded, are hydrophilic in character. Such hydrophilic groups can be charged or uncharged, as is well-known in the art. As specific examples, the R⁵ groups may be selected from hydrogen, optionally substituted (C₁-C₈) alkyl, optionally substituted (C₁-C₈) heteroalkyl, optionally substituted (C₃-C₁₂) cycloalkyl, optionally substituted (C₂-C₁₂) heterocycloalkyl, optionally substituted (C₆-C₁₀) aryl, optionally substituted 5-10 membered heteroaryl, optionally substituted (C₇-C₁₈) arylalkyl and optionally substituted 6-18 membered heteroarylalkyl. The nature of any present substituents can vary widely, as is known in the art. In some embodiments any present substituents are, independently of one another, selected from R^{b}. Each R^{b} is a suitable group independently selected from ═O, —OR^{a}, (C₁-C₃) haloalkyloxy, ═S, —SR^{a}, ═NR^{a}, .═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)R^{a}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{a}, —OC(O)OR^{a}, OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c}; each R^{a} is, independently of the others, selected from hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl; and each R^{c} is, independently of the others, selected from a protecting group and R^{a}, or, alternatively, the two R^{c} bonded to the same nitrogen atom are taken together with that nitrogen atom to form a 5 to 8-membered cycloheteroalkyl or heteroaryl which may optionally include one or more of the same or different additional heteroatoms and which may optionally be substituted with one or more, for example, from one to four, of the same or different R^{a} groups; and each n is, independently of the others, an integer from 0 to 3.

In a specific embodiment, the progroups R are of the formula —CR¹R¹-A-R³, where R³ is selected from —(CH₂)ᵢ—R^{b}, —C(O)R^{a}, —C(O)—(CH₂)ᵢ—R^{b}, —C(O)O—R^{a}, and —C(O)O—(CH₂)ᵢ—R^{b}, where R^{a}, R^{b} and each R¹ independently are as previously defined, and i is an integer ranging from 0 to 6. Specific, non-limiting, examples of exemplary water-solubility increasing progroups include by the way of example and not limitation, hydrophilic groups such as alkyl, arylk, arylalkyl, or cycloheteroalkyl groups substituted with one or more of an amine, alcohol, a carboxylic acid, a phosphorous acid, a sulfoxide, a sugar, an amino acid, a thiol, a polyol, a ether, a thioether and a quaternary amine salt.

One important class of progroups includes progroups that contain a phosphate group, for example, phosphate-containing progroups of the formula —(CR¹R¹)_{y}—O—P(O)(OH)₂, where each R¹ is independently as defined above and y is an integer ranging from 1 to 3,typically 1 or 2. In a specific embodiment, each R¹ is, independently of the others, selected from hydrogen, substituted or unsubstituted lower alkyl, substituted or unsubstituted (C₆-C₁₄) aryl and substituted or unsubstituted (C₇-C₂₀) arylalkyl.

While not intending to be bound by any theory of operation, it is believed that such phosphate-containing progroups R^{p} act as substrates for both alkaline and acid phosphatase enzymes, leading to their removal from the prodrugs under physiological conditions of use. As alkaline phosphatases are abundant in the digestive tract of humans, phosphate-containing progroups R^{p} that can be cleaved in the presence of alkaline phosphatases are particularly suitable for formulating phosphate-containing prodrugs intended for oral administration. Specific examples of phosphate-containing progroups R^{p} suitable for use in prodrugs intended for oral administration include, but are not limited to, groups of the formula —(CR¹R¹)_{y}—O—P(O)(OH)₂ in which each R¹ is, independently of the others, selected from hydrogen and unsubstituted lower alkanyl. Exemplary embodiments of such phosphate-containing progroups include, but are not limited to, —CH₂—O—P(O)(OH)₂ and —CH₂CH₂—O—P(O)(OH)₂. In some embodiments, the progroup is —CH₂—O—P(O)(OH)₂, including ionized forms (e.g., —CH₂—O—P(O)(OH)O⁻ or —CH₂—O—P(O)(O⁻)₂) or salts thereof.

Although phosphate-containing prodrugs suitable for oral administration are of interest, skilled artisans will appreciate that prodrugs including phosphate-containing progroups R^{p} can be administered via other routes of administration, as phosphatases are distributed throughout the body. Thus, the only requirement is that the particular phosphate-containing progroup R^{p} selected should be removable under the conditions of intended use.

In some embodiments of such prodrugs, the phosphorous-containing progroup R^{p} comprises a phosphite group. A specific exemplary embodiment of such phosphite-containing prodrugs includes prodrug compounds in which the progroup R^{p} is of the formula —(CR¹R¹)_{y}—O—P(OH)(OH), where R¹ and y are as previously defined.

In other embodiments of such prodrugs, the phosphorous-containing progroup R^{p} comprises an acyclic phosphate ester or phosphite ester group. Specific exemplary embodiments of such acyclic phosphate ester and phosphite ester prodrugs include progroups R^{p} of the formula —(CR¹R¹)_{y}—O—P(O)(OH)(OR²), —(CR¹R¹)_{y}—O—P(O)(OR²)₂, —(CR¹R¹)_{y}—O—P(OH)(OR²) and —(CR¹R¹)_{y}—O—P(OR²)₂, where R² is selected from substituted or unsubstituted lower alkyl, substituted or unsubstituted (C₆-C₁₄) aryl (e.g., phenyl, naphthyl, 4-lower alkoxyphenyl, 4-methoxyphenyl), substituted or unsubstituted (C₇-C₂₀) arylalkyl (e.g., benzyl, 1-phenylethan-1-yl, 2-phenylethan-1-yl), —(CR¹R¹)_{y}—OR⁵, —(CR¹R¹)_{y}—O—C(O)R⁵, —(CR¹R¹)_{y}—O—C(O)OR⁵, —(CR¹R¹)_{y}—S—C(O)R⁵, —(CR¹R¹)_{y}—S—C(O)OR⁵, —(CR¹R¹)_{y}—NH—C(O)R⁵, —(CR¹R¹)_{y}—NH—C(O)OR⁵ and —Si(R¹)₃, wherein each R⁵ is, independently of the others, selected from hydrogen, unsubstituted or substituted lower alkyl, substituted or unsubstituted (C₆-C₁₄) aryl, and substituted or unsubstituted (C₇-C₂₀) arylalkyl, and R¹ and y are as previously defined.

In still other embodiments, phosphorous-containing prodrugs that include phosphate precursors are prodrugs in which the phosphorous-containing progroup R^{p} comprises a cyclic phosphate ester of the formula:

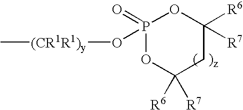

where each R⁶ is, independently of the others, selected from hydrogen and lower alkyl; each R⁷ is, independently of the others, selected from hydrogen, substituted or unsubstituted lower alkyl, substituted or unsubstituted lower cycloheteroalkyl, substituted or unsubstituted (C₆-C₁₄) aryl, substituted or unsubstituted (C₇-C₂₀) arylalkyl and substituted or unsubstituted 5-14 membered heteroaryl; z is an integer ranging from 0 to 2; and R¹ and y are as previously defined.

In still other embodiments, phosphorous-containing prodrugs that include phosphate precursors are prodrugs in which the phosphorous-containing progroup R^{p} comprises a cyclic phosphite ester of the formula

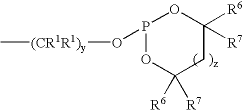

where R⁶, R⁷, R¹, y and z are as previously defined.

In some embodiments, the substituents R⁶ on such cyclic phosphate ester and phosphite ester prodrugs are selected such that the progroup is metabolized in vitro by esterase enzymes. Specific examples of such phosphate ester and phosphite ester progroups include those in which each R⁶ is, independently of the others, selected from hydrogen, lower alkyl, methyl, ethyl and propyl. In some embodiments, such progroups are selected from:

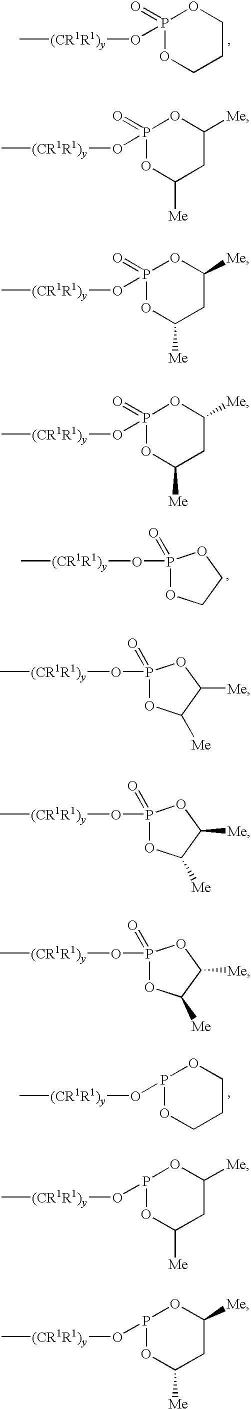

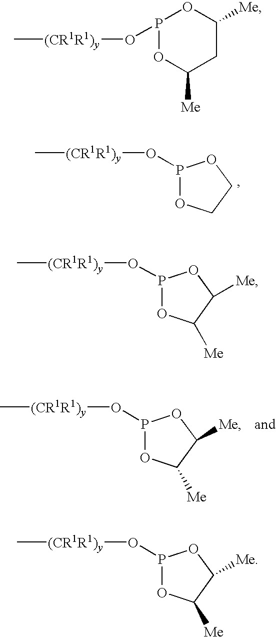

Many of these phosphate esters and phosphite esters are acid label and, when administered orally, metabolize to the corresponding phosphates and phosphites under the acidic conditions of the stomach and/or gut.

Thus, in the phosphorous-containing prodrugs described herein, the identity of the particular phosphorous-containing progroups R^{p} employed can be selected to tailor the prodrugs for particular modes of delivery, etc.

In all of the compounds described herein that include substituent alternatives that may be substituted, such as, for example, some of the substituent alternatives delineated for R¹, R², R⁵, R⁶, and R⁷, the substitutions are typically, independently of one another, selected from amongst the R^{b} groups described above. In a specific embodiment, any present substitutions are, independently of one another, selected from hydroxyl, lower alkoxy, (C₆-C₁₄) aryloxy, lower alkoxyalkyl, methoxymethyl, methoxyethyl, ethoxymethyl, ethoxyethyl and halogen.

Those of skill in the art will appreciate that some of the drug and prodrug compounds of the methods described herein may exhibit the phenomena of tautomerism, conformational isomerism, geometric isomerism and/or optical isomerism. For example, the drug and prodrug compounds may include one or more chiral centers and/or double bonds and as a consequence may exist as stereoisomers, such as double-bond isomers (i.e., geometric isomers), enantiomers and diasteromers and mixtures thereof, such as racemic mixtures. As another example, the drug and prodrug compounds may exist in several tautomeric forms, including the enol form, the keto form and mixtures thereof. As the various compound names, formulae and compound drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the drug and prodrug compounds having one or more of the utilities described herein, as well as mixtures of these various different isomeric forms. In cases of limited rotation around the 2,4-pryimidinediamine core structure, atropisomers are also possible and are also specifically included in the drug and prodrug compounds of the methods herein described.

5.4 Methods of Making the Drug and Prodrug Compounds

Methods for synthesizing the 2,4-pyrimidinediamine drug and prodrug compounds described herein are detailed in U.S. application Ser. No. 11/337,049 filed Jan. 19, 2006 (US2006/0211657 A1), incorporated herein by reference in its entirety, and in particular, in the Examples section 7.1 at paragraphs 247-263 of the printed publication.

The metabolism of a 2,4-pyrimidinediamine prodrug of the instant disclosure is detailed in U.S. application Ser. No. 11/337,049 filed Jan. 19, 2006 (US2006/0211657 A1), at paragraphs 134-142 and 146 of the printed publication. The phosphate-containing prodrug, Compound A, according to the structure illustrated below:

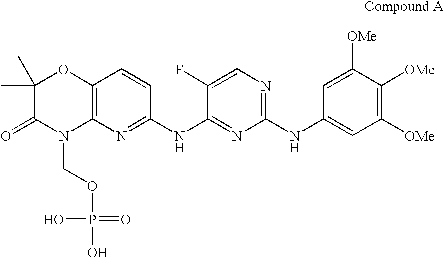

was found to metabolize in vivo to the corresponding active 2,4-pyrimidinediamine compound, Compound 1, illustrated below:

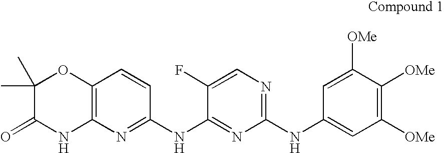

While not intending to be bound by any particular theory, it is believed that this prodrug metabolizes to active Compound 1 via the corresponding hydroxymethylamine intermediate illustrated below:

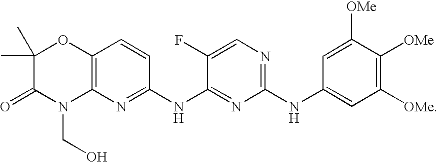

Such hydroxymethylamine compounds are known to be unstable under physiological conditions and various pH ranges where they hydrolyze in vivo to yield formaldehyde and the active drug substance. Based on this observation, it is believed that prodrugs that include hydroxyl “protecting” groups that can be metabolized in vivo, for example by the acidic conditions of the stomach and/or by enzymes present in the digestive tract or other organs and/or tissues or fluids with the body, to yield the hydroxymethylamine intermediate illustrated above will likewise metabolize to the active 2,4 pyrimidinediamine drug.

5.5 In Vitro Uses

To assess the antiproliferative effects of 2,4-pyrimidinediamine drug and prodrug compounds on growth of particular cancer cell lines, the compounds can be administered by contacting cultured tumor cell lines with the compounds. In the context of in vitro assays, administration of the drug or prodrug compound to tumor cells may be simply contacting cells in culture with an amount of the drug or prodrug compound in an amount effective to inhibit proliferation. When the drug compound is supplied in the form of a prodrug compound, the method is carried out under conditions in which the prodrug compound yields the drug compound.

Examples of tumor cell lines derived from human tumors and available for use in the in vivo studies described herein include, but are not limited to, leukemia cell lines: CCRF-CEM, HL-60(TB), K-562, MOLT-4, RPM1-8226, SR, P388 and P388/ADR; non-Small cell lung cancer cell lines: A549/ATCC, EKVX, HOP-62, HOP-92, NCI-H226, NCI-H23, NCI-H322M, NCI-H460, NCI-H522 and LXFL 529; small cell lung cancer cell lines: DMS 114 and SHP-77; colon cancer cell lines: COLO 205, HCC-2998, HCT-1 16, HCT-15, HT29, KM 12, SW-620, DLD-1 and KM20L2; Central Nervous System (CNS) cancer cell lines: SF-268, SF-295, SF-539, SNB-19, SNB-75, U251, SNB-78 and XF 498; melanoma cell lines: LOX I MVI, MALME-3M, M14, SK-MEL-2, SK-MEL-28, SK-MEL-5, UACC-257, UACC-62, RPMI-7951 and M19-MEL; ovarian cancer cell lines: IGROV1, OVCAR-3, OVCAR-4, OVCAR-5, OVCAR-8 and SK-OV-3; renal cancer cell lines: 786-0, A498, ACHN, CAKI-1, RXF 393, SN12C, TK-10, UO-31, RXF-631 and SN12K1; prostate cancer cell lines: PC-3 and DU-145; and breast cancer cell lines: MCF7, NCI/ADR-RES, MDA-MB-231/ATCC, HS 578T, MDA-MB-435, BT-549, T-47D and MDA-MB-468.

5.6 In Vivo Uses

2,4-pyrimidinediamine drug and prodrug compounds can be used to inhibit tumor cell growth in a subject, as a therapeutic approach towards the treatment or prevention of proliferative disorders, such as tumorigenic cancers.

Generally, cell proliferative disorders treatable with the 2,4-pyrimidinediamine drug or prodrug compounds provided herein relate to any disorder characterized by aberrant cell proliferation. These include various tumors and cancers, benign or malignant, metastatic or non-metastatic.
5.6.1 Types of Cancers

A variety of cellular proliferative disorders may be treated using the drug and prodrug compounds via the disclosed methods. In some embodiments, the drug or prodrug compounds are used to treat various cancers in afflicted subjects. Cancers are traditionally classified based on the tissue and cell type from which the cancer cells originate. Carcinomas are considered cancers arising from epithelial cells while sarcomas are considered cancers arising from connective tissues or muscle. Other cancer types include leukemias, which arise from hematopoietic cells, and cancers of nervous system cells, which arise from neural tissue. For non-invasive tumors, adenomas are considered benign epithelial tumors with glandular organization while chondomas are benign tumor arising from cartilage. In the present invention, the described compounds may be used to treat proliferative disorders encompassed by carcinomas, sarcomas, leukemias, neural cell tumors, and non-invasive tumors.

Solid tumor cancers include malignant neoplastic masses of tissue or cancerous neoplasms characterized by the progressive or uncontrolled proliferation of cells. The cells involved in the neoplastic growth have an intrinsic heritable abnormality such that they are not regulated properly by normal methods. Malignant or cancerous neoplasms tend to grow rapidly, spread throughout the body, and recur if removed. The cells of malignant tumors may be well differentiated, but most have some degree of anaplasia. Anaplastic cells tend to be larger than normal and are abnormal, even bizarre, in shape. The nuclei tend to be very large, and irregular, and they often stain darkly. Malignant tumors may be partially encapsulated, but the cells of the cancer can infiltrate and destroy surrounding tissue. Thus, cells from the primary tumor can migrate (metastasize) from the original tumor site and colonize in other tissues. Tumors formed from cells that have spread are referred to as “secondary tumors” and contain cells that are similar to those in the original “primary” tumor. Metastatic tumors typically form by migration of tumor cells from the original tumor site through the blood and lymph system to other tissues.

Specific properties of cancers, such as tissue invasiveness or metastasis, may be targeted using the methods described herein. In some embodiments, the drugs or prodrugs are used to treat solid tumors arising from various tissue types, including, but not limited to, cancers of the bone, breast, respiratory tract (e.g., bladder), brain reproductive organs, digestive tract, urinary tract, eye, liver, skin, head, neck, thyroid, parathyroid, and metastatic forms thereof.

Specific proliferative disorders include the following: a) proliferative disorders of the breast include, but are not limited to, invasive ductal carcinoma, invasive lobular carcinoma, ductal carcinoma, lobular carcinoma in situ, and metastatic breast cancer; b) proliferative disorders of the skin include, but are not limited to, basal cell carcinoma, squamous cell carcinoma, malignant melanoma, and Karposi's sarcoma; c) proliferative disorders of the respiratory tract include, but are not limited to, small cell and non-small cell lung carcinoma, bronchial adema, pleuropulmonary blastoma, and malignant mesothelioma; d) proliferative disorders of the brain include, but are not limited to, brain stem and hyptothalamic glioma, cerebellar and cerebral astrocytoma, medullablastoma, ependymal tumors, oligodendroglial, meningiomas, and neuroectodermal and pineal tumors; e) proliferative disorders of the male reproductive organs include, but are not limited to, prostate cancer, testicular cancer, and penile cancer f) proliferative disorders of the female reproductive organs include, but are not limited to, uterine cancer (endometrial), cervical, ovarian, vaginal, vulval cancers, uterine sarcoma, ovarian germ cell tumor; g) proliferative disorders of the digestive tract include, but are not limited to, anal, colon, colorectal, esophageal, gallbladder, stomach (gastric), pancreatic cancer, pancreatic cancer—Islet cell, rectal, small-intestine, and salivary gland cancers; h) proliferative disorders of the liver include, but are not limited to, hepatocellular carcinoma, cholangiocarcinoma, mixed hepatocellular cholangiocarcinoma, and primary liver cancer; i) proliferative disorders of the eye include, but are not limited to, intraocular melanoma, retinoblastoma, and rhabdomyosarcoma; j) proliferative disorders of the head and cancers include, but are not limited to, laryngeal, hypopharyngeal, nasopharyngeal, oropharyngeal cancers, and lip and oral cancer, squamous neck cancer, metastatic paranasal sinus cancer; k) proliferative disorders of the lymphomas include, but are not limited to, various T cell and B cell lymphomas, non-Hodgkins lymphoma, cutaneous T cell lymphoma, Hodgkins disease, and lymphoma of the central nervous system; l) leukemias include, but are not limited to, acute myeloid leukemia, acute lymphoblastic leukemia, chronic lymphocytic leukemia, chronic myelogenous leukemia, and hair cell leukemia, m) proliferative disorders of the thyroid include thyroid cancer, thymoma, and malignant thymoma; n) proliferative disorders of the urinary tract include, but are not limited to, bladder cancer; o) sarcomas include, but are not limited to, sarcoma of the soft tissue, osteosarcoma, malignant fibrous histiocytoma, lymphosarcoma, and rhabdomyosarcoma.

It is to be understood that the descriptions of proliferative disorders is not limited to the conditions described above, but encompasses other disorders characterized by uncontrolled growth and malignancy. It is further understood that proliferative disorders include various metastatic forms of the tumor and cancer types described herein. The drug and prodrug compounds of the described methods may be tested for effectiveness against these disorders, and a therapeutically effective regimen established. Effectiveness, as further described below, includes reduction or remission of the tumor, decreases in the rate of cell proliferation, or cytostatic or cytotoxic effect on cell growth.
5.6.2 Modes of Administration

In the context of in vivo assays, administration of the 2,4-pyrimidinediamine drug and prodrug compounds of the methods described herein to a subject may be by oral administration, injection, or other suitable means in an amount effective to treat or prevent growth of a solid tumor cancer in a subject. When the drug compound is supplied in the form of a prodrug compound, the method is carried out under conditions in which the prodrug compound yields the drug compound. The cleavage of the promoiety may proceed spontaneously, or it may be catalyzed or induced by another agent endogenous to the conditions of use or supplied exogenously, as described above.

The drugs or prodrugs may be administered per se, or as pharmaceutical compositions, comprising a drug or prodrug.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the drugs or prodrugs may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the drugs or prodrugs in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the drugs or prodrugs may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate, lecithin). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the drug or prodrug, as is well known.

For buccal administration, the compositions may take the form of tablets or lozenges formulated in conventional manner.

For rectal and vaginal routes of administration, the drugs or prodrugs may be formulated as solutions (for retention enemas) suppositories or ointments containing conventional suppository bases such as cocoa butter or other glycerides.

For prolonged delivery, the drugs or prodrugs can be formulated as a depot preparation for administration by implantation or intramuscular injection. The active ingredient may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the drug or prodrug for percutaneous absorption may be used. To this end, permeation enhancers may be used to facilitate transdermal penetration of the drugs or prodrugs. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems may be employed. Liposomes and emulsions are well-known examples of delivery vehicles that may be used to deliver the drugs or prodrugs. Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the drugs or prodrugs. The pack may, for example, comprise metal or plastic foil, such as a blister pack. The pack or dispenser device may be accompanied by instructions for administration.
5.6.3 Formulations

When used to treat or prevent a solid tumor cancer, the 2,4-pyrimidinediamine compounds of the disclosure may be administered singly, as mixtures of one or more active compounds or in mixture or combination with other agents useful for treating cancer and/or the symptoms associated with cancer. The 2,4-pyrimidinediamine compounds of the disclosure may also be administered in mixture or in combination with agents useful to treat other disorders or maladies, such as steroids, membrane stablizers.

Pharmaceutical compositions comprising the 2,4-pyrimidinediamine drug and prodrug compounds used in the methods herein disclosed may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the 2,4-pyrimidinediamine compounds into preparations which can be used pharmaceutically (see Remingtons 's Pharmaceutical Sciences, 15 th Ed., Hoover, J. E. ed., Mack Publishing Co. (2003)

The 2,4-pyrimidinediamine drug and prodrug compounds may be formulated in the pharmaceutical compositions per se, or in the form of a hydrate, solvate, N-oxide or pharmaceutically acceptable salt. Such salts may be derived from acids or bases, as is well-known in the art. Typically, such salts are more soluble in aqueous solutions than the corresponding free acids and bases, but salts having lower solubility than the corresponding free acids and bases may also be formed. Such salts include salts suitable for pharmaceutical uses (“pharmaceutically-acceptable salts”), salts suitable for veterinary uses, etc. In some embodiments, the salt is a pharmaceutically acceptable salt. Generally, pharmaceutically acceptable salts are those salts that retain substantially one or more of the desired pharmacological activities of the parent compound and which are suitable for administration to humans.
5.6.4 Dosages

The 2,4-pyrimidinediamine drug and prodrug compounds, or compositions thereof, will generally be used in an amount effective to achieve the intended result, for example in an amount sufficient to treat a specified disorder or disease or one or more of its symptoms and/or to prevent the occurrence of the disease or disorder.

The compound(s) may be administered therapeutically to achieve therapeutic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also includes halting or slowing the progression of the disease, regardless of whether improvement is realized.

In reference to tumorigenic proliferative disorders, a pharmaceutically or therapeutically effective amount comprises an amount sufficient to, among other things, cause the tumor to shrink or to decrease the growth rate of the tumor, or to cause the tumor cells to undergo a process of apoptotic cell death. Effectiveness, as further described below, includes reduction or remission of the tumor, decreases in the rate of cell proliferation, or cytostatic or cytotoxic effect on cell growth.

The amount of compound administered will depend upon a variety of factors, including, for example, the particular indication being treated, the mode of administration, the severity of the indication being treated and the age and weight of the patient, the bioavailability of the particular active compound, etc. Determination of an effective dosage is well within the capabilities of those skilled in the art.

Effective dosages may be estimated initially from in vitro assays. For example, an initial dosage for use in animals may be formulated to achieve a circulating blood or serum concentration of active compound that is at or above an IC₅₀ (the concentration inhibitory to growth in 50% of the population, also referred to herein as the GI₅₀) of the particular compound as measured in an in vitro assay, such as the in vitro assays described in the Examples section. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pagamonon Press, and the references cited therein.

Initial dosages may also be estimated from in vivo data, such as animal models. Animal models useful for testing the efficacy of compounds to treat or prevent the various diseases described above are well-known in the art. Dosage amounts will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma levels of the compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) may be determined using standard pharmaceutical procedures. The dose ratio between toxic and therapeutic (or prophylactic) LD₅₀/ED₅₀ effect is the therapeutic index (LD₅₀ is the dose lethal to 50% of the population and ED₅₀ is the dose therapeutically effective in 50% of the population). Compounds(s) that exhibit high therapeutic indices are preferred.
5.6.5 Combination Therapies

The compounds of the present disclosure may be used alone, in combination, or as an adjunct to, or in conjunction with, other established antiproliferative therapies or with cytotoxic agents. Thus, the compounds of the present disclosure may be used with traditional cancer therapies, such as ionization radiation in the form of γ-rays and x-rays, delivered externally or internally by implantation of radioactive compounds, and as a follow-up to surgical removal of tumors. The compounds of the present disclosure and the other therapeutic agent may be administered simultaneously, sequentially, by the same route of administration, or by different routes.

Various chemotherapeutic agents may be used in combination with the 2,4-pyrimidinediamine compounds provided herein to treat inhibit tumor cell proliferation. These chemotherapeutic agents may be general cytotoxic agents or target a specific cellular molecule. Various classes of cancer chemotherapeutic agents include, among others, antimetabolites, agents that react with DNA (e.g., alkylating agents, coordination compounds, etc.), inhibitors of transcription enzymes, topoisomerase inhibitors, DNA minor-groove binding compounds, antimitotic agents (e.g., vinca alkyloids), antitumor antibiotics, hormones, and enzymes. Exemplary alkylating agents include, by way of example and not limitation, mechlorothamine, cyclophosphamide, ifosfamide, melphalan, chlorambucil, ethyleneimines, methylmelamines, alkyl sulfonates (e.g., busulfan), and carmustine. Exemplary antimetabolites include, by way of example and not limitation, folic acid analog methotrexate; pyrimidine analogs fluorouracil, cytosine arabinoside; and purine analogs mecaptopurine, thioguanine, and azathioprine. Exemplary vinca alkyloids include, by way of example and not limitation, vinblastine, vincristine, paclitaxel, and colchicine. Exemplary antitumor antibiotics include, by way of example and not limitation, actinomycin D, daunorubicin, and bleomycin. An exemplary enzyme effective as anti-neoplastic agent is L-asparaginase. Exemplary coordination compounds include, by way of example and not limitation, cisplatin and carboplatin. Exemplary hormones and hormone related compounds include, by way of example and not limitation, adrenocorticosteroids prednisone, and dexamethasone; aromatase inhibitors amino glutethimide, formestane, and anastrozole; progestin compounds hydroxyprogesteron caproate, medroxyprogesterone; and anti-estrogen compound tamoxifen. Exemplary topoisomerase inhibitors include, by way of example and not limitation, amsacrine (m-AMSA); mitoxantrone, topotecan, irinotecan, and camptothecin. Various derivative anti-neoplastic agents that combine more than one anticancer activity may be used. For instance, NSC₂₉₀₂₀₅ is a combination compound incorporating d-lactam derivative of androsterone and an alkylating agent based on N,N-bis(2-chloroethyl)aniline (Trafalis et al., 2005, Br. J. Haematol. 128(3):343-50).

These and other useful anti-cancer compounds are described in Merck Index, 13th Ed. (O'Neil M. J. et al., ed) Merck Publishing Group (2001) and Goodman and Gilmans The Pharmacological Basis of Therapeutics, 10th Edition, Hardman, J. G. and Limbird, L. E. eds., pg. 1381-1287, McGraw Hill, (1996), both of which are incorporated by reference herein.

Additional antiproliferative compounds useful in combination with the 2,4-pyrimidinediamine compounds described herein include, by way of example and not limitation, antibodies directed against growth factor receptors (e.g., anti-Her2); cytokines such as interferon-α and interferon-γ, interleukin-2,and GM-CSF; and antibodies for cell surface markers (e.g., anti-CTLA-4. anti-CD20 (rituximab); anti-CD33). When antibodies against cell surface markers are used, a chemotherapeutic agent may be conjugated to it for delivering the agent to the tumor cell. Suitable conjugates include radioactive compounds (e.g., radioactive metal bound to a antibody conjugated chelator), cytotoxic compounds, and drug activating enzymes (e.g., allinase, peptidases, esterases, catalytic antibodies, etc.) (see, e.g., Arditti et al., 2005, Mol. Cancer Therap. 4(2):325-331; U.S. Pat. No. 6,258,360; incorporated herein by reference)

In some embodiments, the 2,4-pyrimidinediamine compounds provided herein may be used with a kinase inhibitor that targets an oncogenic kinase. In some embodiments, the kinase inhibitor is an inhibitor of Abl kinase. For example, chronic myelogenous leukemia is a myeloid neoplasm characterized by malignant proliferation of leukemic stem cells in the bone marrow. The majority of chronic myelogenous leukemia is associated with a cytogenetic abnormality defined by a reciprocal translocation t(9;22)(q34;q11). This chromosomal aberration results in generation of a BCR/ABL fusion protein with activated kinase activity. Inhibitors of the fusion protein kinase activity are effective in treating chronic myelogenous leukemia although resistant forms may develop upon continued treatment. Use of the 2,4-pyrimidinediamine compounds provided herein in combination of Abl kinase inhibitors may lessen the chances of resistant cells by targeting a cellular process different than that targeted by the kinase inhibitor alone. An exemplary Abl kinase inhibitor is 2-phenylaminopyrimidine, also known as imatinib mesylate and Gleevec®. Thus, in some embodiments, the 2,4-pyrimidinediamine compounds provided herein may be used in combination with Abl kinase inhibitor 2-phenylaminopyrimidine and its derivatives. In other embodiments, the kinase inhibitor may be pyridol[2-3-d]pyrimidine and its derivatives, which was originally identified as inhibitors of Src kinase. In yet further embodiments, the kinase inhibitor is tyrphostins and its derivatives (e.g., adaphostin) which affects the association of the kinase with its substrates. Other kinase inhibitor compounds will be apparent to the skilled artisan.

As further described herein, the administration of other chemotherapeutic agents may be done in the form of a composition, or administered adjunctively in combination with the 2,4-pyrimidinediamine compounds provided herein. When provided adjunctively, the chemotherapeutic agents may be administered simultaneously with or sequentially with administration of the 2,4-pyrimidinediamine compound.

### 6. EXAMPLES

### Example 1

### The Drug Compounds Inhibit Proliferation of Tumor Cells Without Cytotoxicity to Normal Cells

The 2,4-pyrimidinediamine drug and prodrug compounds of the methods herein disclosed were synthesized using methods described in U.S. application Ser. No. 11/337,049 filed Jan. 19, 2006 (US2006/0211657 A1), in particular, in the Examples section 7.1 at paragraphs 247-263 of the printed publication. Salts of the compounds were prepared using standard techniques, and used in screening tumor cell lines for antiproliferative activity.

In exemplary embodiments of the methods for inhibiting tumor cell proliferation using a besylate salt of 2,4-pyrimidinediamine drug Compound 1,the GI₅₀, TGI and LC₅₀ values of the drug were determined using standard in vitro antiproliferation assays. “GI₅₀” refers to the concentration of compound at which inhibition of growth of 50% of the population of cells being assayed was observed. “TGI” refers to the concentration of compound at which total inhibition of growth of cells being assayed was observed. “LC₅₀” refers to the concentration of compound which resulted in lethality in 50% of the population of cells being assayed. The effects of 2,4-pyrimidinediamine drug Compound 1 (besylate salt) on tumor cell proliferation are illustrated in Table 1,below. A blank indicates that the drug compound was not tested against the specified cell line.

TABLE 1
Effect of drug on cancer cell proliferation

| Cancer | Panel/Cell | | | |
|---|---|---|---|---|
| Type | Line | G1₅₀ | TGI | LC₅₀ |
| Leukemia | CCRF-CEM | | >1.00E−4 | >1.00E−4 |
| | HL-60(TB) | >1.00E−4 | >1.00E−4 | >1.00E−4 |
| | K-562 | >1.00E−4 | >1.00E−4 | >1.00E−4 |
| | MOLT-4 | 4.82E−6 | >1.00E−4 | >1.00E−4 |
| | RPM1-8226 | >1.00E−4 | >1.00E−4 | >1.00E−4 |
| | SR | 3.56E−6 | >1.00E−4 | >1.00E−4 |
| Non-Small | A549/ATCC | | >1.00E−4 | >1.00E−4 |
| Cell Lung | EKVX | 1.66E−6 | >1.00E−4 | >1.00E−4 |
| Cancer | HOP-62 | 5.37E−7 | >1.00E−4 | >1.00E−4 |
| | HOP-92 | 4.10E−7 | | >1.00E−4 |
| | NCI-H226 | 4.97E−7 | | >1.00E−4 |
| | NCI-H23 | | >1.00E−4 | >1.00E−4 |
| | NCI-H322M | >1.00E−4 | >1.00E−4 | >1.00E−4 |
| | NCI-H460 | | >1.00E−4 | >1.00E−4 |
| | NCI-H522 | | >1.00E−4 | >1.00E−4 |
| Colon | COLO 205 | 7.26E−6 | >1.00E−4 | >1.00E−4 |
| Cancer | HCC-2998 | >1.00E−4 | >1.00E−4 | >1.00E−4 |
| | HCT-116 | | >1.00E−4 | >1.00E−4 |
| | HCT-15 | | >1.00E−4 | >1.00E−4 |
| | HT29 | 2.19E−6 | >1.00E−4 | >1.00E−4 |
| | KM12 | 3.55E−7 | >1.00E−4 | >1.00E−4 |
| | SW-620 | | >1.00E−4 | >1.00E−4 |
| CNS | SF-268 | 1.70E−6 | >1.00E−4 | >1.00E−4 |
| Cancer | SF-295 | | >1.00E−4 | >1.00E−4 |
| | SF-539 | 6.05E−7 | | >1.00E−4 |
| | SNB-19 | >1.00E−4 | >1.00E−4 | >1.00E−4 |
| | SNB-75 | 8.71E−7 | >1.00E−4 | >1.00E−4 |
| | U251 | | >1.00E−4 | >1.00E−4 |
| Melanoma | LOX I MVI | 1.13E−6 | 1.13E−6 | >1.00E−4 |
| | MALME-3M | >1.00E−4 | >1.00E−4 | >1.00E−4 |
| | M14 | >1.00E−4 | >1.00E−4 | >1.00E−4 |
| | SK-MEL-2 | >1.00E−4 | >1.00E−4 | >1.00E−4 |
| | SK-MEL-28 | >1.00E−4 | >1.00E−4 | >1.00E−4 |
| | SK-MEL-5 | >1.00E−4 | >1.00E−4 | >1.00E−4 |
| | UACC-257 | >1.00E−4 | >1.00E−4 | >1.00E−4 |
| | UACC-62 | | >1.00E−4 | >1.00E−4 |
| Ovarian | IGROV1 | 7.96E−7 | >1.00E−4 | >1.00E−4 |
| Cancer | OVCAR-3 | >1.00E−4 | >1.00E−4 | >1.00E−4 |
| | OVCAR-4 | >1.00E−4 | >1.00E−4 | >1.00E−4 |
| | OVCAR-5 | >1.00E−4 | >1.00E−4 | >1.00E−4 |
| | OVCAR-8 | >1.00E−4 | >1.00E−4 | >1.00E−4 |
| | SK-OV-3 | 4.29E−7, | >1.00E−4 | >1.00E−4 |
| Renal | 786-0 | 8.73E−7 | >1.00E−4 | >1.00E−4 |
| Cancer | A498 | 3.60E−7 | | >1.00E−4 |
| | ACHN | 1.24E−6 | >1.00E−4 | >1.00E−4 |
| | CAKI-1 | 5.15E−8 | 2.44E−6 | >1.00E−4 |
| | RXF 393 | 1.96E−7 | 7.74E−7 | >1.00E−4 |
| | SN12C | 6.98E−7 | >1.00E−4 | >1.00E−4 |
| | TK-10 | | >1.00E−4 | >1.00E−4 |
| | UO-31 | 5.96E−7 | >1.00E−4 | >1.00E−4 |
| Prostate | PC-3 | 3.07E−6 | >1.00E−4 | >1.00E−4 |
| Cancer | DU-145 | | >1.00E−4 | >1.00E−4 |
| Breast | MCF7 | 2.72E−6 | >1.00E−4 | >1.00E−4 |
| Cancer | NCI/ADR-RES | >1.00E−4 | >1.00E−4 | >1.00E−4 |
| | MDA-MB- | | >1.00E−4 | >1.00E−4 |
| | 231/ATCC | | | |
| | HS 578T | 1.19E−6 | >1.00E−4 | >1.00E−4 |
| | MDA-MB-435 | 3.62E−6 | >1.00E−4 | >1.00E−4 |
| | BT-549 | 2.43E−6 | >1.00E−4 | >1.00E−4 |
| | T-47D | | >1.00E−4 | >1.00E−4 |

As shown in Table 1, antiproliferative effects of the 2,4-pyrimidinediamine drug Compound 1 (besylate salt) were observed in several cell lines, and in particular in most renal tumor cell lines. As shown in the column labeled LC₅₀, these antiproliferative effects were apparently not attributable to cytotoxicity and/or cell death.

### Example 2

### The Drug Compounds Exhibit Activity in Xenograft Studies Using A498 Renal Carcinoma Cells

NCR nu/nu female mice (8-9 weeks old, Harlan Laboratories, Madison, Wis.) were injected subcutaneously on the right hind flank with 5×10⁷ A498 renal carcinoma cells in a 50:50 mixture of cells in PBS and Matrigel® (Cat# 356234, LDEV negative, BD Biosciences, Boston, Mass.). Tumor growth was determined by measuring the tumor in two dimensions (L×W) with electronic calipers (Ultra Cal IV, Fred V Fowler Co, Newton, Mass.). Volume was calculated using Study Director Animal Study Management Software (Studylog Systems, South San Francisco, Calif.) as follows: V=(L×W²)/2,where L and W are the tumor length and width, respectively. Animals were randomized by stratified tumor size and treatment was initiated when the average tumor size was approximately 82 mm³ (Study Day 0, 17 days post-injection). Test compound was administered ad libitum in the feed as a formulation of 0, 0.5, 2, or 3 g Compound A per kg of AIN-76A rodent diet (Research Diets, Inc, New Brunswick, N.J.). Animals were weighed and tumors measured twice weekly from the day of cell injection to study termination.

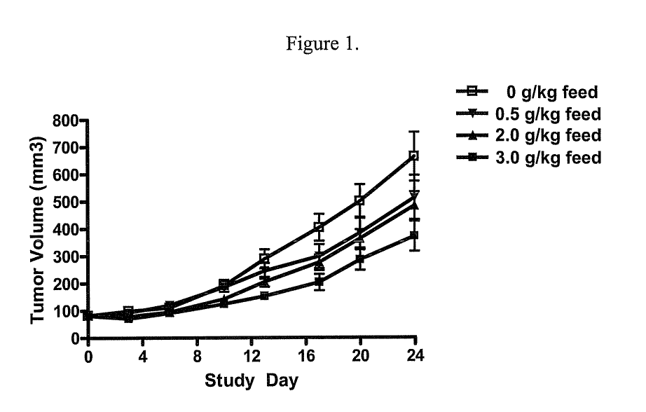

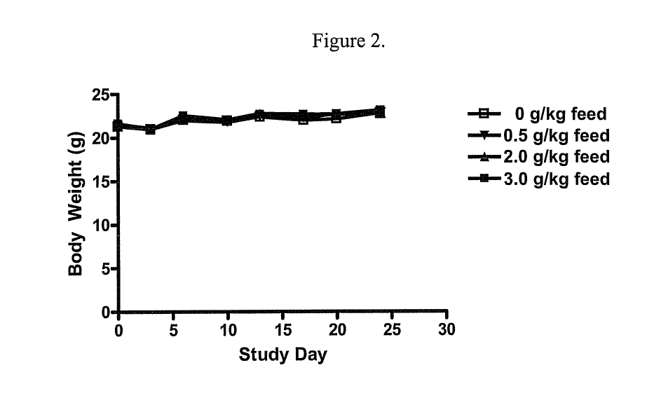

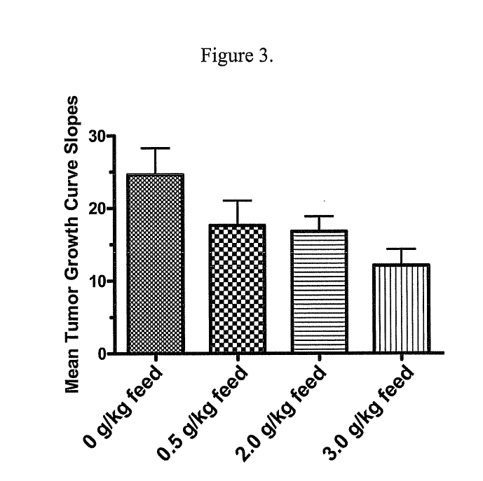

The animals implanted with A498 responded to treatment with Compound A administered in the feed, which was available ad libitum (FIG. 1). The mean tumor volume of vehicle vs. drug-treated animals after 24 days of treatment at the high dose (3.0 g Compound A/kg feed) was 665 vs. 372 mm³ (p<0.05). The median change in tumor volume in the treated group relative to the median change in tumor volume in the control group expressed as a percent (% T/C) was calculated to be 35%. A dose-dependent effect on tumor growth was observed. Animals fed a diet with 2.0 g Compound A/kg feed also demonstrated less reduction in tumor growth, and by Day 23 the mean tumor volume was 485 mm³ compared with 665 mm in the control group (p>0.05, %T/C=62%). Similar results were seen with the low dose group. Mean body weights were not different among the groups (FIG. 2), indicating a higher dose level of Compound A could be used to treat animals with tumors. Mean slopes are shown in FIG. 3.

### Example 3

### The Drug Compounds Exhibit Activity in Xenograft Studies Using RXF-393 Renal Carcinoma Fragments

RXF-393 kidney tumor fragments were implanted intrarenally into Nu/nu mice. On days 5-20 post-implantation animals were fed the Research Diet chow containing Compound A at the following dose levels: 0 g/kg (control), 0.5 g/kg (expected dose 75 mg/kg/day), 2.0 g/kg (expected dose 300 mg/kg/day), or 3.0 g/kg (expected dose 450 mg/kg/day). Tumors were allowed to grow orthotopically, body weights were measured twice weekly, and the animals were sacrificed on Day 23. Both the implanted kidney and the unimplanted kidney were weighed, and the tumor weight is expressed as the difference in kidney weights for each animal.

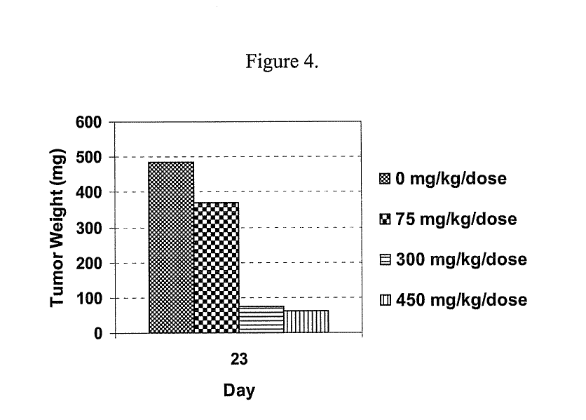

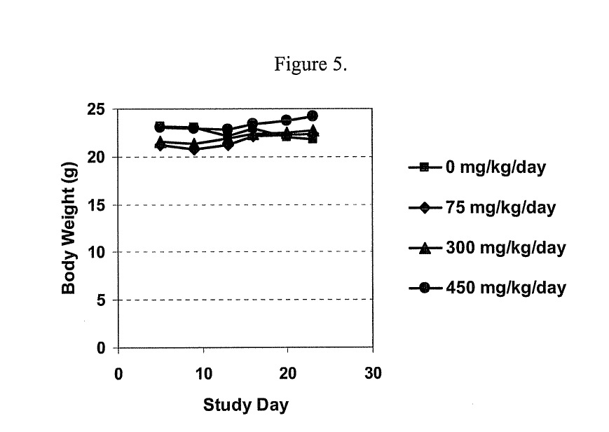

The animals implanted orthotopically with RXF-393 responded to treatment with Compound A. Tumor weights were reduced significantly in the groups treated with 3 g Compound A/kg food (450 mg/kg/dose) and 2 g Compound A/kg food (300 mg/kg/dose) compared with vehicle, p<0.0001 and p<0.0006,respectively. Following 16 days of treatment, the median tumor weight was 63 mg and 74 mg for the high- (3 g/kg) and mid- (2 g/kg) dose groups, while the median tumor weight of the vehicle group was 485 mg, representing a nearly 10-fold reduction in tumor weight. Animals in the low dose group (75 mg/kg/dose, 0.5 g Compound A/kg food) showed a slight reduction in median tumor weight of approximately 25%, compared with vehicle. Median tumor weights for each group are shown in FIG. 4. Mean body weights were similar among the groups (FIG. 5), indicating a higher dose level of Compound A could be used to treat animals with tumors.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of inhibiting proliferation of a tumor cell in a subject, comprising the step of administering to the subject an amount of a prodrug compound according to structural formula (I): 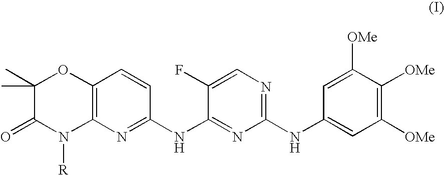
or a pharmaceutically acceptable salt, hydrate, or N-oxide thereof, wherein R is a progroup selected from the group consisting of acid labile hydroxyalkyl-containing progroup, an acid labile thio containing progroup, an acid labile amino containing progroup, an acid labile phosphate containing progroup, and salts thereof.

2. The method of claim 1, in which R is a group of the formula —(CR¹R¹)_{y}—O—P(O)(OH)₂,
wherein each R¹ is independently selected from hydrogen optionally substituted lower alkyl, optionally substituted (C₆-C₁₄) aryl and optionally substituted (C₇-C₂₀) arylalkyl; where the optional substituents are, independently of one another, selected from hydroxyl, lower alkoxy, (C₆-C₁₄) aryloxy, lower alkoxyalkyl, and halogen, or, alternatively, two R¹ bonded to the same carbon atom are taken together with the carbon atom to which they are bonded to form a cycloalkyl group containing from 3 to 8 carbon atoms;
and y is an integer ranging from 1 to 3.

3. The method of claim 2, in which R is —CH₂—O—P(O)(OH)₂, including ionized forms or salts thereof.

4. The method of any one of claims 2-3, in which the tumor cell is a renal tumor cell.

5. The method of any one of claims 2-3, in which the prodrug compound is administered in the form of a pharmaceutical composition.

6. The method of any one of claims 2-3, in which the prodrug compound is administered orally or intravenously.

7. The method of any one of claims 2-3, in which the subject is human.

8. A method of treating a solid tumor cancer in a subject, comprising administering to the subject an amount of a compound according to structural formula (I) effective to treat the solid tumor cancer: 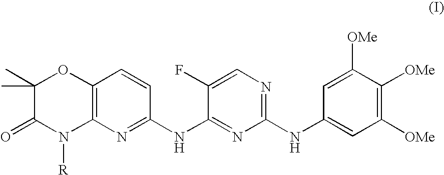
or a pharmaceutically acceptable salt, hydrate, or N-oxide thereof, wherein R is a progroup selected from the group consisting of acid labile hydroxyalkyl-containing progroup, an acid labile thio containing progroup, an acid labile amino containing progroup, an acid labile phosphate containing progroup, and salts thereof.

9. The method of claim 8, in which the progroup is a group of the formula —(CR¹R¹)_{y}—O—P(O)(OH)₂.
wherein each R¹ is independently selected from hydrogen optionally substituted lower alkyl, optionally substituted (C₆-C₁₄) aryl and optionally substituted (C₇-C₂₀) arylalkyl; where the optional substituents are, independently of one another, selected from hydroxyl, lower alkoxy, (C₆-C₁₄) aryloxy, lower alkoxyalkyl, and halogen, or, alternatively, two R¹ bonded to the same carbon atom are taken together with the carbon atom to which they are bonded to form a cycloalkyl group containing from 3 to 8 carbon atoms;
and y is an integer ranging from 1 to 3.

10. The method of claim 8, in which the progroup is —CH₂—O—P(O)(OH)₂, including ionized forms or salts thereof.

11. The method of any one of claims 8-10, in which the compound is administered in the form of a pharmaceutical composition.

12. The method of any one of claims 8-10, in which the compound is administered orally or intravenously.

13. The method of any one of claims 8-10, in which the solid tumor cancer is selected from renal cell carcinoma, ovarian carcinoma, kidney carcinoma, clear cell carcinoma of kidney, renal cell adenocarcinoma, ovarian adenocarcinoma, colon adenocarcinoma, lung adenocarcinoma, large cell lung carcinoma, squamous cell carcinoma of the lung, mesothelioma, and glioma.

14. The method of any one of claims 8-10, in which the solid tumor cancer is renal cell carcinoma and/or renal cell adenocarcinoma.

15. The method of any one of claims 8-10, in which the subject is a human.
